(19) United States
(12) Patent Application Publication
(10) Pub. No.: US 2021/0316498 A1
(43) Pub. Date: Oct. 14, 2021

(54) SYSTEMS AND METHODS FOR 3D PRINTING OF PROTEINS

(71) Applicant: Trustees of Tufts College, Medford, MA (US)

(72) Inventors: David L. Kaplan, Medford, MA (US); Xuan Mu, Medford, MA (US)

(21) Appl. No.: 17/269,582

(22) PCT Filed: Aug. 20, 2019
(86) PCT No.: PCT/US2019/047359

§ 371 Date: Feb. 19, 2021

Related U.S. Application Data
(60) Provisional application No. 62/720,016, filed on Aug. 20, 2018.

(51) Int. Cl. B29C 64/106; C09D 11/04; C07K 14/435; C12N 5/073; B33Y 10/00; B33Y 70/00; B29C 64/30; B33Y 40/20

(52) U.S. Cl. CPC B29C 64/106; C09D 11/04; C07K 14/43586; C12N 5/0605; B33Y 10/00; B29K 2105/0032; B29C 64/30; B33Y 40/20; C12N 2533/50; B29K 2089/00; B33Y 70/00

(57) ABSTRACT

Three-dimensional printing methods and systems for forming a three-dimensional protein article are disclosed. The methods and systems involve selecting article formation parameters, such as protein ink parameters, solvent bath parameters, shear force parameters, and mapping parameters. After these parameters are selected, the methods and systems iteratively introduce protein ink into a solvent bath via a three-dimensional printing outlet. The result is a three-dimensional protein article. One exemplary protein is silk fibroin. Further processing can be done, such as drying the article.

### CROSS-REFERENCE TO RELATED APPLICATIONS

This application is related to, claims priority to, and incorporated herein by reference for all purposes U.S. Provisional Patent Application No. 62/720,016, filed Aug. 20, 2018.

### STATEMENT REGARDING FEDERALLY SPONSORED RESEARCH

This invention was made with government support under grant EB002520 awarded by the National Institutes of Health. The government has certain rights in the invention.

### BACKGROUND

Protein is one of the most essential and superior structural materials in nature, from cellular cytoskeleton to spider silk, highly promising in a wide range of applications including regenerative medicine, drug delivery, implantable devices, bioelectronics and biophotonics. The exceptional features of proteins, often unavailable in synthetic materials, closely relate to the manufacturing process through a controlled hierarchical self-assembly with molecule and nanoscale precision. However, it has proven to be challenging to achieve the self-assembly with the similar precision in various manufacturing techniques and three-dimensional (3D) printing, which largely rely on high energy beam/laser, high temperature, organic solvents and chemical crosslinking, thus suffering from deteriorated strength, compromised biocompatibility and limited shape complexity.

Currently, there is a need in the art for the development of manufacturing and printing techniques that maximize biocompatibility, degradability of printed structures, and minimize costs by eliminating additives and extra steps.

### SUMMARY OF THE DISCLOSURE

The present disclosure addresses the aforementioned drawbacks by providing compositions and manufacturing techniques that are based on the self-assembly of protein molecules. In general, the present disclosure provides a bio-ink composition, biocompatible 3D printed structures, a 3D printing system, and methods of using the same.

In an aspect, the present disclosure provides a three-dimensional printing method for making a three-dimensional silk article. The method includes: a) selecting an article formation parameter set including one or more silk fibroin solution parameters, one or more solvent bath parameters, one or more shear force parameters, and one or more mapping parameters; and b) iteratively introducing a silk fibroin solution into a solvent bath via a three-dimensional printing outlet, thereby forming the three-dimensional silk article. The silk fibroin solution, the solvent bath, and control of the iteratively introducing at based on the article formation parameter set. Step b) is free of photo-cross-linkers, chemical cross-linkers, and organic solvents. The method can optionally include removing the three-dimensional silk article from the solvent bath and drying the three-dimensional silk article.

One advantage of the present disclosure is to provide biocompatible 3D printed structures and 3D printing techniques that do not rely on chemical or photocrosslinking compounds, additives (e.g., organic solvents), and no external stimuli (e.g., heat). Eliminating organic solvents and chemical/photocrosslinking compounds from the manufacturing process increases the biocompatibility and degradability of printed structures, and reduces costs by eliminating additives and process steps, thereby simplifying the manufacturing process.

Another advantage of some aspects of the present disclosure is the use of induced self-assembly of protein molecules to hierarchical structures with molecule and nanoscale precision. Owing to the precise assembly, the printed structures have little defects and mechanical strength that can be maximized. Overall, this present disclosure provides a greener and more energy effective manufacturing technique for printing 3D protein structures that improves over conventional techniques that rely on high energy beam/laser, high temperature, organic solvents and chemical crosslinking (photopolymerization), thus suffering from deteriorated strength, compromised biocompatibility and limited shape complexity.

These and other advantages and features of the present invention will become more apparent from the following detailed description of the preferred embodiments of the present invention when viewed in conjunction with the accompanying drawings.

### BRIEF DESCRIPTION OF THE DRAWINGS

FIG. 1a is a schematic of 3D printing in accordance with an aspect of the present disclosure, that uses a rationally devised aqueous salt bath to direct molecular assembly for constructing 3D ordered and hierarchical structures. G, glycine; A, alanine; S, serine.

FIG. 1b is an example of a printed monolithic proteinaceous structure. Scale bar, 5 mm.

FIG. 1c is an example of a printed monolithic proteinaceous structure. Scale bar, 5 mm.

FIG. 1d is an example of a printed monolithic proteinaceous structure. Scale bar, 5 mm.

FIG. 2: Rheological and structural characterization of 3D prints: a, Viscosity-Shear rate profile of protein inks at different concentrations and fitted curves. b, FEA simulations of protein ink in the nozzle. c, solvent induced increase of storage modulus. d, FTIR spectrum of freeze dried protein ink, transparent 3D prints and degummed silk fibers. e, Transmittance of wet and dry 3D prints. f, morphology of 3D printings at surface and cross-section.

FIG. 3: Biofunctions and degradation of 3D prints. a, Uniaxial tensile stress-strain curve of prints designed in a dog-bone shape. Inset Scale bar, 10 mm. b, prints incubated in saline for 2 and 6 days placed on a metal rod with a diameter of 1.26 mm. Scale bar, 3 mm. c, a print in a rectangle lattice repeatedly stretched. Scale bar, 5 mm. d, comparison of this work to other 3D printed biopolymers in terms of modulus and strength. e, Schematics of bioactive protein ink doped with functional materials. f, 3D printed lattice doped with HRP glowing after adding the substrate (Luminol and H2O2). g, 3D prints doped with Quantum dots (emission 560 nm). Scale bar, 5 mm. h, Degradation profile and typical images.

FIG. 4: Fabrication of microfluidic chips with valves: a, Confocal image of the endothelialization of a 4-layer protein lattice. Scale bar, 100 μm. b, schematic and image of a Y-shape microfluidic chip. c, Cross-section of the main channel indicated in the blue dash line. d, multi-material printing with two protein inks in a 4-layer lattice. e, Time-lapse images of air flowing inside microfluidic channel to show airtightness. Narrow arrow shows the position of air-liquid interface. Wide arrow shows the direction of the air.

FIG. 5: 3D printed monolithic protein-based structures. a, a pyramid in 32-layer. b, a half sphere in 23-layer. c, a radical circle in 8-layer. d, a hollow star in 13-layer. Bright field image shows its bottom. e, a square in 40-layer. f. a rectangle lattice in 40-layer. Scale bars, 1 mm.

FIG. 6: The measurement of flow rate and the calculation of shear rate. a, One example of the droplet formation under 70 kPa. b, the droplet was assumed as a perfect sphere with a diameter (d) to calculate the change of volume (V) along with time to obtain the flow rate (Q). The flow of the ink inside nozzle in a diameter (D) is assumed as a simple capillary flow, and thus, the shear rate (γ) at the wall can be calculated. c, The measured volumetric flow rate and the calculated shear rate is plotted against pressure of compressed air. Red line indicates linear fitting.

FIG. 7: a, Dynamic time sweep of protein inks at 30% and 27% for 15 minutes. b, Dynamic strain sweep of protein ink at 30% for determining Linear Viscoelastic Region (LVR) from 0.5% to 4%.

FIG. 8: The deconvolution and quantitative analysis of secondary structures (β-sheet, β-turn and Random coil) in amide I peak of protein ink, prints and degummed silk fibers.

FIG. 9: a and b, detailed geometry of printed dog-bone structures for tensile tests. Red dash line indicates the cross-section in b. c, video screenshots at strain of 0% and 270%. d, comparison on modulus, toughness, strength and extensibility with different incubation times in saline solution and post-processing with methanol. e, cross-section of a printed single filament by breaking in liquid nitrogen. f, cross-section caused by tensile breaking.

FIG. 10: Structural characterizations of printing ink and 3D prints. a. Viscosity-shear rate profile of the protein ink, Herschel-Bulkley (HB) model and FEA simulation. Red hollow diamonds indicate printing pressure of 210 kPa. Inset, simulated shear rate vs. printing pressure. b. Oscillatory time sweep of the protein ink to show the dynamics of molecular assembly. An arrow indicates the addition of solutions with different potassium concentrations. c. Fourier transform infrared spectroscopy (FTIR) of the protein ink and the print. The numbers in brackets indicate semi-quantitative content of β-sheet. f Typical uniaxial tensile stress-strain curves of single fibers from a seven-layer 3D print (more in Figure S4d). g. The ultimate tensile toughness of this work is compared with that of photo-crosslinked silk fibroin, ionic-crosslinked polysaccharide (alginate), synthetic composite biopolymers (hydroxyapatite-polycaprolactone, HA/PCL and, hydroxyapatite/polylactic-co-glycolic acid, HA/PLGA). All samples are tested in wet. Error bar represents standard deviation with three repeats. h. Transmittance curves and images of 3D prints. A 3D printed membrane with ˜0.1 mm thickness is used for transmittance measurement. Grey dash line indicates the boundary.

FIG. 11a shows profiles and images of in vitro enzymatic degradation of 39-layer rectangle lattices processed by lyophilization or critical point drying (CPD).

FIG. 11b shows a schematic of a 3D printed four-layer lattice with spatially programmed rhodamine B (RhB).

FIG. 12: Single-step 3D printing of bifurcated microfluidic channels. a. Images and design of 3D printed microfluidic channel. A 25 Gauge needle, slightly larger than the inner diameter of the channel, can be conveniently inserted for tubing. b. Confocal image of the cross-section of the main channel.

FIG. 13: a and b, the semi-quantitative deconvolution of secondary structures in amide I band (1,580-1,720 cm-1) for the protein ink and the 3D print, respectively. The range of gaussian peak of each structure is assigned as follows: 1) β-sheet (1,618-1,629 cm⁻¹), 2) random coil (1,630-1,657 cm⁻¹), 3) helix (1,658-1,667 cm⁻¹) and 4) β-turn (1,670-1,696 cm⁻¹). Gray line, red dash line and blue dash line indicates experimental spectrum, cumulative fit peak and peaks of each structures, respectively. c and d, Raman spectrum and mapping of the printed filament. The content of β-sheet, indicated by the peak area between 1664-1668 cm⁻¹, is homogeneous across the cross-section.

FIG. 14: a, Illustrations and photos of a seven-layer 3D print containing multiple spanning filaments. A single filament is under uniaxial tensile test. b, SEM images of a 3D printed filament after tensile test show the aligned nanofibrous (˜200 nm) cross-section. c, 3D printed four-layer lattice is stretched without damage or delamination (Movie S4). d, Stress-Strain curves of three dry samples and three wet samples for calculating average mechanical properties shown in FIG. 2G. e, Comparison on tensile strength, tensile toughness and elastic modulus with other typical biomaterials listed in Table S2. The ultimate tensile toughness of this work is superior to that of other 3D printed proteins and polymers.

FIG. 15 is a flowchart of a method in accordance with an aspect of the present disclosure.

### DETAILED DESCRIPTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices and methods relating to modifying biological molecules are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

It should be appreciated that compositions that undergo some chemical transformation during their use can be described in various ways. For instance, dissolving NaCl in water can be described as water having an NaCl concentration or water having a concentration of Na+ and Cl− ions. In the present disclosure, components of chemical compositions can be described either as the form they take prior to any chemical transformation or the form they take following the chemical transformation. If there is any ambiguity to a person having ordinary skill in the art, the assumption should be that the component is being described in the context of the particular composition being described (i.e., if describing a finished product or an intermediary after a given chemical transformation, then the chemically transformed entity is being described, and if describing a starting product or intermediary prior to the chemical transformation, then the untransformed entity is being described.

Described herein is a biomimetic process and systems for the 3D printing of proteins based on self-assembly of proteins. The advantages of this process are the direct use of proteins in water, without any crosslinkers or additives required for the process, and the aqueous ambient conditions to permit doping with bioactive components that retain function in the printed structures. Further, the printed proteinaceous structures demonstrated superior mechanical strength, optical transparency, the ability to form complicated 3D geometries and cytocompatibility; all demonstrated with the fabrication of functional microfluidic chips. In one aspect, the 3D printing method described herein employs shear stress and solvent effects to induce the self-assembly of protein molecules at multiple scales, which allows a 3D printing-compatible phase-transition from soluble ink to insoluble filament. The 3D printed structures may be optimized to feature a desirable combination of macroscopic physical properties (mechanical strength, elasticity and optical transparency) and biocompatibility (endothelization, controlled degradation and preservation of labile enzymes).

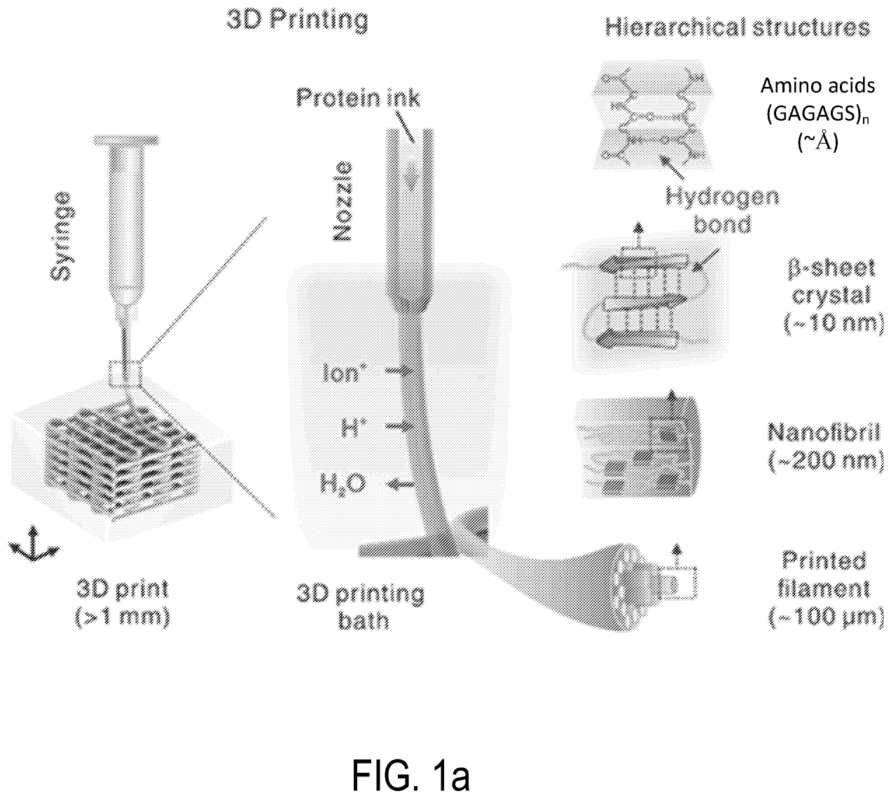

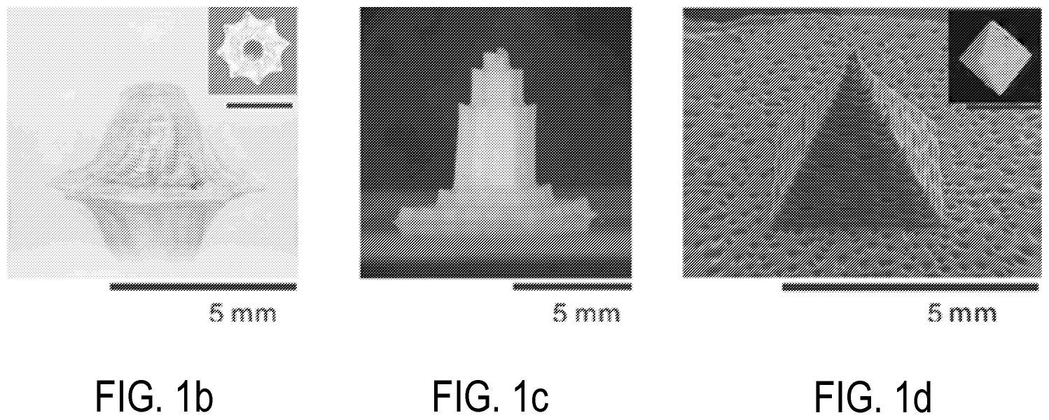

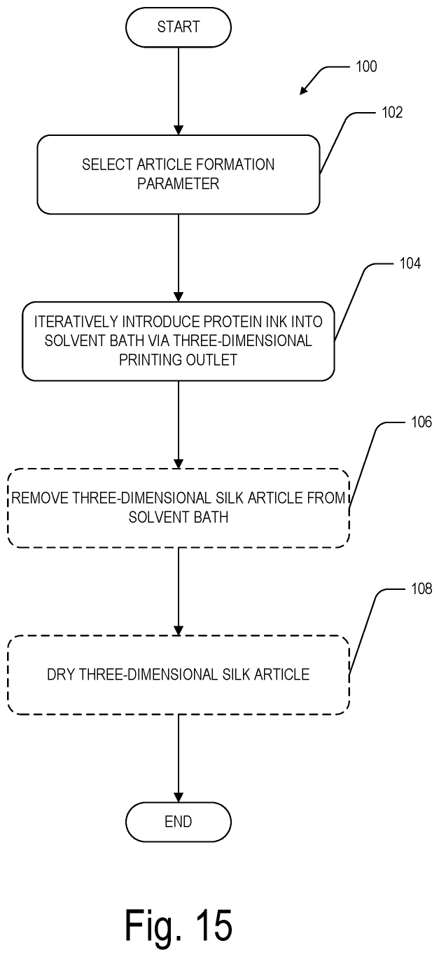

Referring to FIG. 15, the present disclosure provides a three-dimensional printing method 100 for making a three-dimensional silk article. At process block 102, the method 100 includes selecting an article formation parameter. The article formation parameter includes one or more silk fibroin solution parameters, one or more solvent bath parameters, one or more shear force parameters, and one or more mapping parameters. At process block 104, the method 100 includes iteratively introducing a silk fibroin solution into a solvent bath via a three-dimensional printing outlet, thereby forming the three-dimensional silk article. The silk fibroin solution, the solvent bath, and control of the iteratively introducing of process block 104 are based on the article formation parameter set. The iteratively introducing and thereby forming of process block 104 can be free of photo-crosslinkers, chemical cross-linkers, and/or organic solvents. At optional process block 106, the method 100 optionally includes removing the three-dimensional silk article from the solvent bath. At optional process block 108, the method 100 optionally includes drying the three-dimensional silk article. The drying can be freeze drying, critical point drying, or other drying methods understood by those having ordinary skill in the art to be suitable for use with the method 100. FIG. 1a is a schematic representation of the method 100. The method 100 is described in the context of silk fibroin, but is also applicable to other proteins as will be understood by those having ordinary skill in the art. In the context of other proteins, the silk fibroin solution of the method 100 is replaced with a protein ink including the protein of interest.

Before the three-dimensional silk article has been dried or in the absence of drying, the wet article can have impressive elastic modulus or Young's modulus, ultimate stress (i.e., ultimate tensile strength), tensile toughness, ultimate strain (i.e., tensile strain), beta-sheet content, beta-turn content, transmittance, and/or combinations thereof.

In some cases, the wet article can have an elastic modulus or Young's modulus of at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 105 MPa, at least 110 MPa, at least 115 MPa, at least 120 MPa, at least 125 MPa, at least 130 MPa, at least 140 MPa, at least 150 MPa, at least 160 MPa, at least 170 MPa, at least 180 MPa, at least 190 MPa, at least 200 MPa, or greater. In some cases, the wet article can have an elastic modulus or Young's modulus of at most 1000 MPa, at most 950 MPa, at most 900 MPa, at most 850 MPa, at most 800 MPa, at most 750 MPa, at most 700 MPa, at most 650 MPa, at most 600 MPa, at most 550 MPa, at most 500 MPa, at most 450 MPa, at most 400 MPa, at most 350 MPa, at most 325 MPa, at most 300 MPa, at most 275 MPa, at most 250 MPa, at most 225 MPa, at most 200 MPa, at most 175 MPa, at most 150 MPa, at most 125 MPa, at most 100 MPa, or lower.

In some cases, the wet article can have an ultimate stress (i.e., an ultimate tensile strength) of at least 0.1 MPa, at least 0.5 MPa, at least 1 MPa, at least 5 MPa, at least 10 MPa, at least 25 MPa, at least 30 MPa, at least 35 MPa, at least 40 MPa, at least 45 MPa, at least 50 MPa, at least 55 MPa, at least 60 MPa, at least 65 MPa, at least 70 MPa, at least 75 MPa, or greater. In some cases, the dried article can have an ultimate stress of at most 100 MPa, at most 95 MPa, at most 90 MPa, at most 85 MPa, at most 80 MPa, at most 75 MPa, at most 70 MPa, at most 65 MPa, at most 60 MPa, at most 55 MPa, at most 50 MPa, at most 45 MPa, at most 40 MPa, at most 35 MPa, at most 30 MPa, at most 25 MPa, or lower.

In some cases, the wet article can have an ultimate strain (i.e., an ultimate tensile strain) or extensibility of at least 35.0%, at least 40.0%, at least 45.0%, at least 50.0%, at least 55.0%, at least 60.0%, at least 65.0%, at least 70.0%, at least 75.0%, at least 80.0%, at least 85.0%, at least 90.0%, at least 95.0%, at least 100.0%, at least 105.0%, at least 110.0%, at least 115.0%, at least 120.0%, at least 125.0%, at least 130.0%, at least 140.0%, at least 150.0%, at least 160.0%, at least 170.0%, at least 180.0%, at least 190.0%, at least 200.0%, at least 250.0%, or greater. In some cases, the dried article can have an ultimate strain of at most 500.0%, at most 450.0%, at most 400.0%, at most 350.0%, at most 300.0%, at most 250.0%, at most 200.0%, at most 150.0%, at most 140.0%, at most 130.0%, at most 120.0%, at most 110.0%, at most 100.0%, at most 95.0%, at most 90.0%, at most 85.0%, at most 80.0%, at most 75.0%, or lower.

In some cases, the wet article can include silk fibroin having a β-sheet content of less than 10%, less than 9%, less than 8%, less than 7.5%, less than 7%, less than 6.5%, less than 6%, less than 5.5%, less than 5%, less than 4.5%, less than 4%, less than 3.5%, less than 3%, less than 2.5%, less than 2%, less than 1.5%, less than 1%, less than 0.5%, or lower. In some cases, the dried article can include silk fibroin having a β-turn content of greater than 5%, greater than 10%, greater than 15%, greater than 20%, greater than 25%, greater than 30%, or higher.

In some cases, the wet article can have a visible light transmittance of at least 80.0%, at least 82.5%, at least 85.0%, at least 87.5%, at least 90.0%, at least 91.0%, at least 92.0%, at least 93.0%, at least 94.0%, at least 95.0%, at least 96.0%, at least 97.0%, at least 98.0%, at least 99.0%, or greater. In other cases, the dried article can have a lower visible light transmittance, including cases where a dye or opacifying agent has been added to the ink prior to printing.

After the three-dimensional silk article has been dried, the dried article can have impressive elastic modulus or Young's modulus, ultimate stress (i.e., ultimate tensile strength), tensile toughness, ultimate strain (i.e., tensile strain), beta-sheet content, beta-turn content, transmittance, and/or combinations thereof.

In some cases, the dried article can have an elastic modulus or Young's modulus of at least 0.1 GPa, at least 0.2 GPa, at least 0.3 GPa, at least 0.4 GPa, at least 0.5 GPa, at least 0.6 GPa, at least 0.7 GPa, at least 0.8 GPa, at least 0.9 GPa, at least 1.0 GPa, at least 1.1 GPa, at least 1.2 GPa, at least 1.3 GPa, at least 1.4 GPa, at least 1.5 GPa, at least 1.6 GPa, at least 1.7 GPa, at least 1.8 GPa, at least 1.9 GPa, at least 2.0 GPa, at least 2.25 GPa, at least 2.5 GPa, at least 2.75 GPa, at least 3.0 GPa, at least 3.5 GPa, at least 4.0 GPa, at least 4.5 GPa, at least 5.0 GPa, or greater. In some cases, the dried article can have an elastic modulus of at most 10.0 GPa, at most 9.5 GPa, at most 9.0 GPa, at most 8.5 GPa, at most 8.0 GPa, at most 7.5 GPa, at most 7.0 GPa, at most 6.75 GPa, at most 6.5 GPa, at most 6.25 GPa, at most 6.0 GPa, at most 5.75 GPa, at most 5.5 GPa, at most 5.25 GPa, at most 5.0 GPa, at most 4.75 GPa, at most 4.5 GPa, at most 4.25 GPa, at most 4.0 GPa, at most 3.75 GPa, at most 3.5 GPa, at most 3.25 GPa, at most 3.0 GPa, at most 2.75 GPa, at most 2.5 GPa, at most 2.25 GPa, at most 2.0 GPa, at most 1.75 GPa, at most 1.5 GPa, at most 1.25 GPa, at most 1.0 GPa, or lower.

In some cases, the dried article can have an ultimate stress (i.e., an ultimate tensile strength) of at least 1 MPa, at least 5 MPa, at least 10 MPa, at least 25 MPa, at least 30 MPa, at least 35 MPa, at least 40 MPa, at least 45 MPa, at least 50 MPa, at least 55 MPa, at least 60 MPa, at least 65 MPa, at least 70 MPa, at least 75 MPa, at least 100 MPa, or greater. In some cases, the dried article can have an ultimate stress of at most 500 MPa, at most 450 MPa, at most 400 MPa, at most 350 MPa, at most 300 MPa, at most 250 MPa, at most 200 MPa, at most 150 MPa, at most 125 MPa, at most 100 MPa, at most 95 MPa, at most 90 MPa, at most 85 MPa, at most 80 MPa, at most 75 MPa, at most 70 MPa, at most 65 MPa, at most 60 MPa, or lower.

In some cases, the dried article can have an ultimate strain (i.e., an ultimate tensile strain) or extensibility of at least 0.1%, at least 0.2%, at least 0.3%, at least 0.4%, at least 0.5%, at least 0.6%, at least 0.7%, at least 0.8%, at least 0.9%, at least 1.0%, at least 1.25%, at least 1.50%, at least 1.75%, at least 2.0%, at least 2.5%, at least 3.0%, at least 3.5%, at least 4.0%, at least 4.5%, at least 5.0%, at least 6.0%, at least 7.0%, at least 8.0%, at least 9.0%, at least 10.0%, or greater. In some cases, the dried article can have an ultimate strain of at most 50.0%, at most 45.0%, at most 40.0%, at most 35.0%, at most 30.0%, at most 25.0%, at most 20.0%, at most 15.0%, at most 14.0%, at most 13.0%, at most 12.0%, at most 11.0%, at most 10.0%, at most 9.5%, at most 9.0%, at most 8.5%, at most 8.0%, at most 7.5%, at most 7.0%, at most 6.5%, at most 6.0%, at most 5.5%, at most 5.0%, at most 4.5%, at most 4.0%, at most 3.5%, at most 3.0%, at most 2.5%, or lower.

In some cases, the dried article can include silk fibroin having a β-sheet content of greater than 5%, greater than 10%, greater than 15%, greater than 20%, greater than 25%, greater than 30%, greater than 35%, greater than 37.5%, greater than 40%, greater than 41%, greater than 42%, greater than 43%, greater than 44%, greater than 45%, greater than 46%, greater than 47%, greater than 48%, greater than 49%, greater than 50%, greater than 51%, greater than 52%, greater than 53%, greater than 54%, greater than 55%, greater than 60%, greater than 65%, greater than 70%, greater than 75%, greater than 80%, greater than 85%, greater than 90%, or higher. In some cases, the dried article can include silk fibroin having a β-turn content of less than 15%, less than 14%, less than 13%, less than 12%, less than 11%, less than 10%, less than 9%, less than 8%, less than 7.5%, less than 7%, less than 6.5%, less than 6%, less than 5.5%, less than 5%, less than 4.5%, less than 4%, less than 3.5%, less than 3%, less than 2.5%, less than 2%, less than 1.5%, less than 1%, less than 0.5%, or lower.

In some cases, the dried article can have a visible light transmittance of at least 80.0%, at least 82.5%, at least 85.0%, at least 87.5%, at least 90.0%, at least 91.0%, at least 92.0%, at least 93.0%, at least 94.0%, at least 95.0%, at least 96.0%, at least 97.0%, at least 98.0%, at least 99.0%, or greater. In other cases, the dried article can have a lower visible light transmittance, including cases where a dye or opacifying agent has been added to the ink prior to printing.

The 3D printing method of the present disclosure can include selecting an article formation parameter set including one or more insoluble ink parameters, one or more solvent bath parameters, one or more shear force parameters, and one or more mapping parameters. In one aspect, the insoluble ink can be a silk fibroin solution. The silk fibroin solution parameter can include the group consisting of silk fibroin concentration, silk fibroin molecular weight distribution, and combinations thereof. The silk fibroin solution can have a silk fibroin concentration between 10 wt % and 40 wt %, or between 15 wt % and 40 wt %, or between 20 wt % and 40 wt %, or between 25 wt % and 40 wt %, or between 30 wt % and 40 wt %. The silk fibroin solution can have a silk fibroin concentration between 10 wt % and 40 wt %, or between 10 wt % and 35 wt %, or between 10 wt % and 30 wt %, or between 10 wt % and 25 wt %, or between 10 wt % and 20 wt %. The molecular weight distribution of the silk fibroin can be at least 10 kDa, 20 kDa, 30 kDa, 40 kDa, 50 kDa, 60 kDa, 70 kDa, 80 kDa, 90 kDa, or another minimum value understood by those having ordinary skill in the art to be a useful value for preparing a silk fibroin solution for 3D printing. The molecular weight cut-off can be at most 450 kDa, 400 kDa, 350 kDa, 300 kDa, 250 kDa, 200 kDa, 150 kDa, 100 kDa, 90 kDa, or another maximum value understood by those having ordinary skill in the art to be a useful value for preparing a silk fibroin solution for 3D printing.

In one aspect, the solvent bath parameters can include a parameter selected from the group consisting of a chemical composition of the solvent bath, a soak time, pH, temperature and combinations thereof.

In one aspect, the chemical composition of the solvent bath comprises one or more salts. In one non-limiting example, the solvent bath can contain a total salt concentration of at least 500 mM, or at least 1 M, or at least 2 M, or at least 3 M, or at least 4 M, or at least 5 M. In another non-limiting example, the solvent bath can contain a total salt concentration of at most 10 M, at most 8 M, at most 7 M, at most 6 M, or at most 5 M, or at most 4 M, or at most 3M, or at most 2 M, or at most 1 M. In another non-limiting example, the one or more salts are selected from the group consisting of sodium chloride, dipotassium phosphate, ammonium sulfate, and combinations thereof. In some cases, the solvent bath include sodium chloride at a concentration of 5.0 M or lower. In some cases, the solvent bath includes dipotassium phosphate at a concentration of 2.0 M or lower. In some cases, the solvent bath includes ammonium sulfate at a concentration of 2.25 M or lower. In one specific example, the solvent bath is a solution containing 0.5 M dipotassium phosphate and 4 M sodium chloride. In some cases, the solvent bath includes potassium ions and sodium ions. In some cases, the solvent bath includes potassium ions in a concentration of between 0.1 M and 2.0 M. In some cases, the solvent bath includes sodium ions in a concentration of between 3.0 M and 5.0 M. In comes cases, the solvent bath has an osmolarity of at least 8 M, at least 9 M, at least 10 M, or at least 12 M. In some cases, the solvent bath has an osmolarity of at most 20 M, at most 16 M, or at most 12 M.

In another aspect, the pH of the solvent can be at least 2.5, 3, 3.5, 4, 4.5, 5, 5.5, 6, 6.5, 7, or anther minimum value understood by those having ordinary skill in the art to be a useful value of preparing a solvent bath for 3D printing of silk fibroin. In another aspect, the pH of the solvent can be at most 7.5, 7, 6.5, 6, 5.5, 5, or anther maximum value understood by those having ordinary skill in the art to be a useful value for preparing a solvent bath for 3D printing of silk fibroin. In some cases, the solvent bath has a pH of between 4 and 7 or between 5 and 7.

In one aspect, the solvent bath is at a temperature of 20° C., or 25° C., or 30° C., or 35° C., or 40° C., or a temperature between these values. In some cases, the solvent bath is at room temperature.

In one embodiment, the silk fibroin solution or protein ink is an aqueous solution. In some cases, the silk fibroin solution or protein ink includes silk fibroin or other protein in an amount by weight of at least 10%, at least 15%, at least 20%, at least 25%, at least 30%. In some cases, the silk fibroin solution or protein ink includes silk fibroin or other protein in an amount by weight of at most 40%, at most 37.5%, at most 35%, at most 32.5%, at most 30%, or at most 25%.

The 3D printing method of the present disclosure can further include iteratively introducing a silk fibroin solution into a solvent bath via a three-dimensional printing outlet, thereby forming the three-dimensional silk article, wherein the silk fibroin solution, the solvent bath, and control of the iteratively introducing are based on the article formation parameter set.

The mapping parameters of the 3D printing method of the present disclosure can include locations and volumes at which the silk solution is introduced though the fine nozzle into the saline bath. In one aspect, the mapping parameter can include a nozzle movement speed. In another aspect, the one or more mapping parameter are selected by converting a three-dimensional digital image file into the one or more mapping parameters through a computer, wherein the computer is in electronic communication with a printer.

Suitable printers may include a commercial extrusion-based 3D printer from Cellink, Sweden. In one aspect, the extrusion rate of the 3D printer is controlled by compressed air and valves to enter shear thinning region, which is obtained in a viscosity-shear rate profile. In one aspect, the shear rate during extrusion is higher than that of natural, in vivo silk spinning which is between 1-10 seconds⁻¹, or between 2-10 seconds⁻¹, or between 4-10 seconds⁻¹, or between 6-10 seconds⁻¹, or between 8-10 seconds⁻¹. In some aspects, the protein ink is loaded into a syringe (3 ml) equipped with a fine nozzle with an inner diameter of between 5 μm to 500 μm, or between 50 μm to 100 μm, or between 100 μm to 250 μm, or between 200 μm to 300 μm, or between 300 μm to 400 μm, or between 400 μm to 450 μm, or any other combination of lower and upper bounds between this list. The printing head may be controlled to move at a speed from 0.1 to 10 mm/s, or from 0.2 to 10 mm/s, or from 0.5 to 10 mm/s, or from 1 to 10 mm/s, or from 3 to 10 mm/s, or from 5 to 10 mm/s. The prints may be harvested from the saline solution after 1-5 days, or 2-5 days, or 3-5 days, or 4-5 days for freeze drying or critical point drying, followed by a variety of characterizations of morphology, mechanics, optics and biocompatibility.

The silk fibroin solution of the 3D printing method of the present disclosure can further include an additive. In one aspect, the additive can be selected from the group consisting of a mammalian cell, a bioactive molecule, an antibody, an antibiotic, a nanoparticle, dyes, and combinations thereof. In one non-limiting example, the mammalian cell may comprise a human umbilic vein endothelial cell (HUVEC). In another non-limiting example, the bioactive molecule may comprise horseradish peroxidase. In another non-limiting example, the antibiotic may comprise ampicillin. In another non-limiting example, the nanoparticle can be selected from the group consisting of a gold, quantum dots, and combinations thereof. In another non-limiting example, the dye may comprise a fluorescent dye.

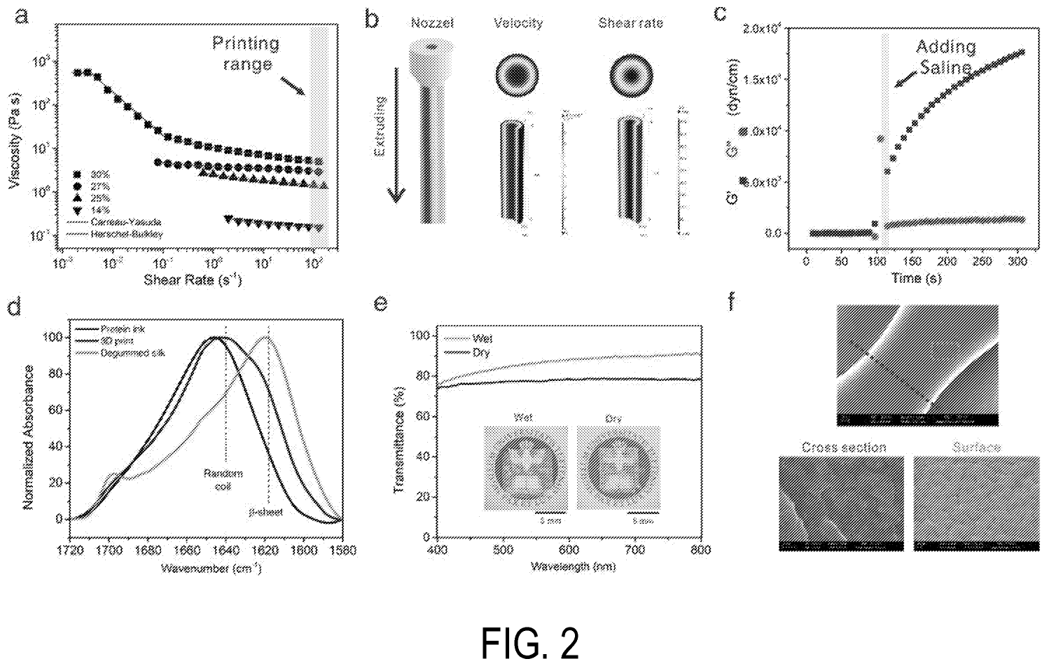

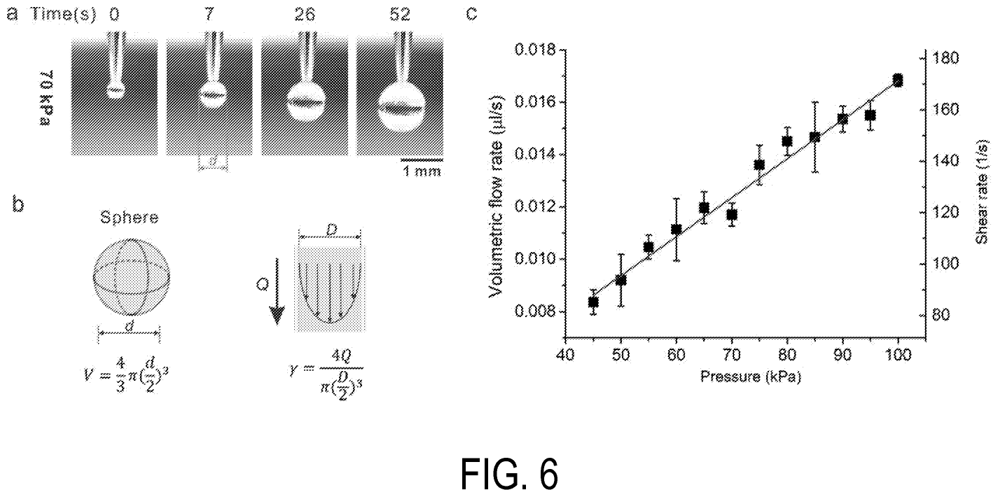

FIGS. 2a-c show a non-limiting example of rheology characterization and Finite Element Analysis (FEA) Simulation of protein ink having 14-30 wt % silk fibroin protein. In one aspect, the shear rate during printing ranges from 80 to 180 s⁻¹, as shown in FIG. 6, which is in the shear thinning region. This is above the in vivo shear rate (1-10 s⁻¹) of silk spinning in spiders.

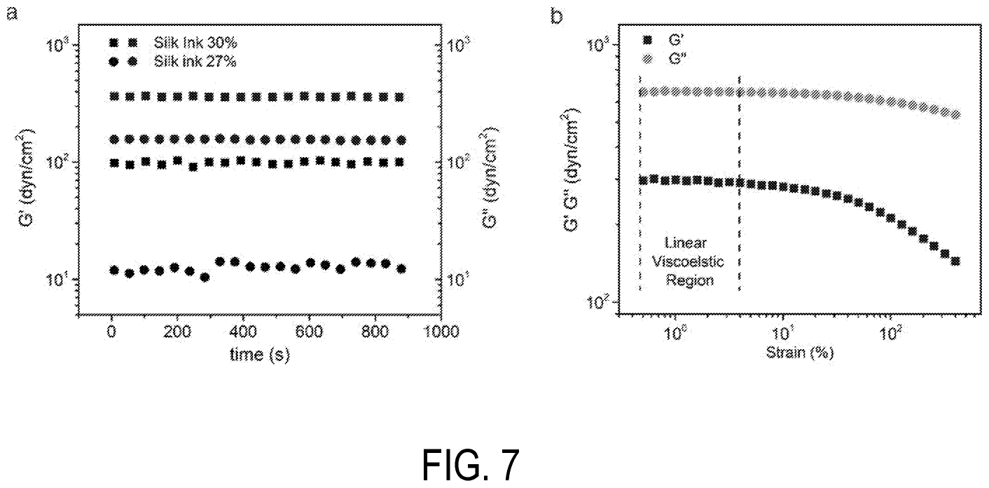

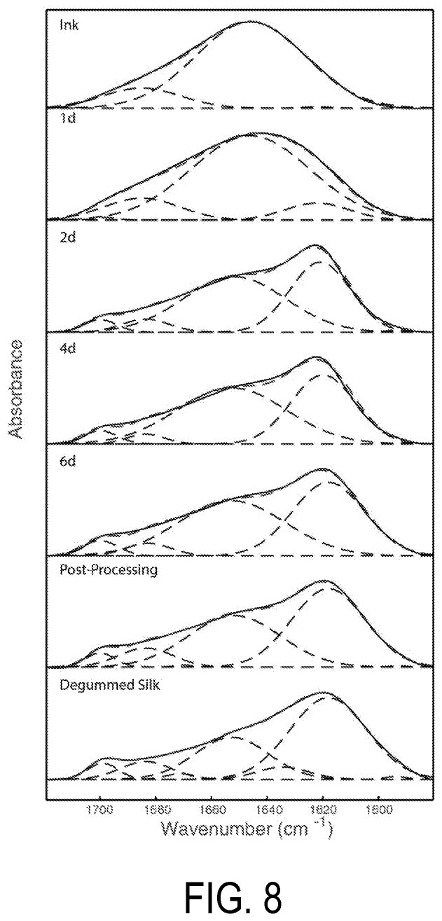

In some aspects, the addition of saline solution increases the storage modulus (G′) of the protein ink by more than a thousand-fold, as shown in FIG. 2b and FIG. 7. The high salt concentration, lower pH value, and high osmolarity of the saline solution mimics salting-out effects, acidification, and water removal, respectively, in the silk spinning process. Fourier Transform Infrared Spectroscopy (FTIR) demonstrated that the amide I peak gradually shifted from 1649 cm⁻¹ to 1621 cm⁻¹, indicating a significant conformational change from random coil to β-sheet (FIG. 2c, FIG. 8, and Table 1 below).

TABLE 1

| | | | | Young's | Ultimate | | |
|---|---|---|---|---|---|---|---|
| | β-sheet | β-turn | Random | Modulus | Strength | Extensibility | Toughness |
| | (%) | (%) | coil (%) | (GPa) | (MPa) | (%) | (KPa) |
| Protein Ink | 0.0358 | 14.1319 | 85.5096 | — | — | — | — |
| 3D prints | | | | | | | |
| Degummed | 58.4754 | 9.2770 | 32.2476 | 9.5 ± 0.2 | 370 ± 20 | 23 ± 1 | |
| silk fiber ^{a} | | | | | | | |

^{a} Materials Letters 60 (2006) 919

The transmittance of 3D print is above 80% over visual range (FIG. 2e). The surface and cross-section of one protein filament demonstrate a highly packed morphology (FIG. 2f).

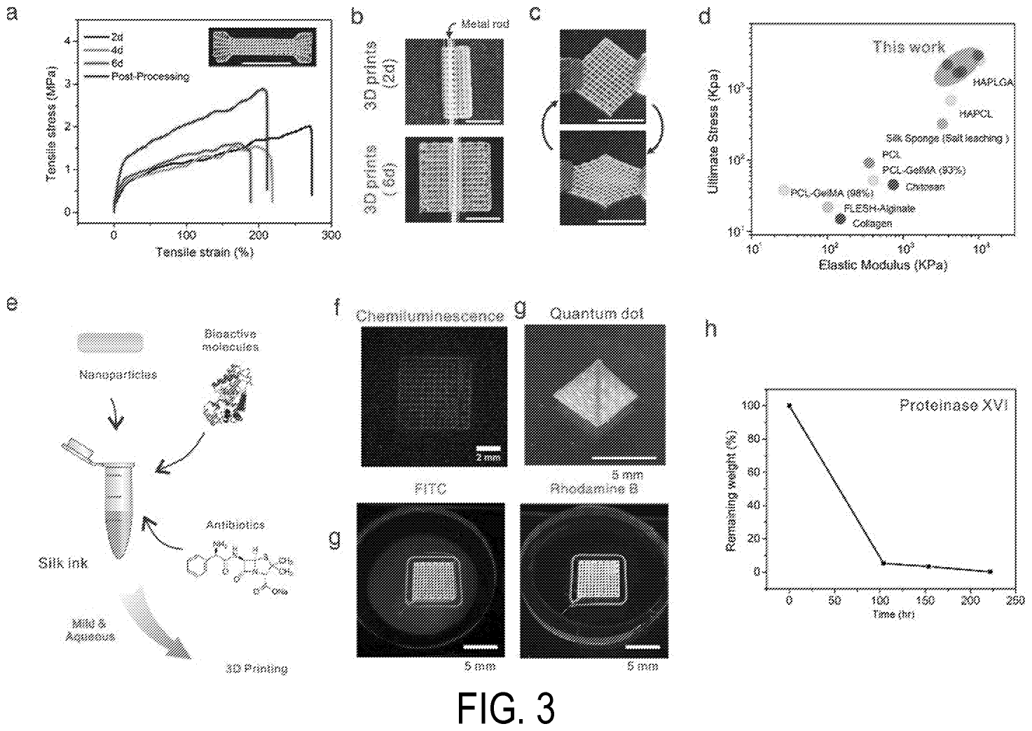

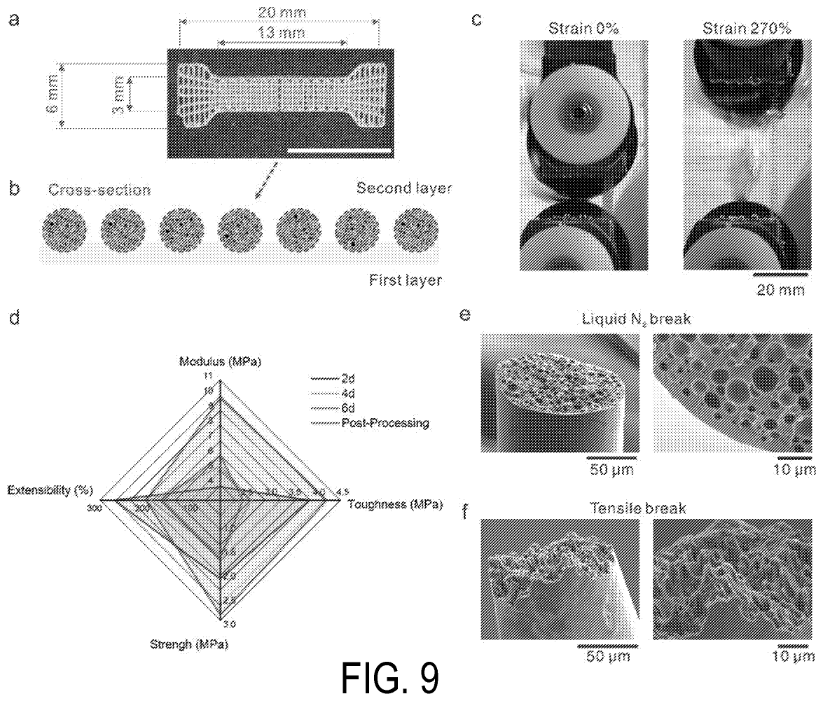

The 3D protein prints demonstrated robust mechanical properties and structural integrity. A two-layer structure in a dog-bone shape was designed and printed for uniaxial tensile tests (FIG. 3a and FIG. 9). The prints may exhibit a high degree of extensibility, from 170% to 270%. Further, it was possible to control mechanical properties by tuning the incubation time in the saline solution post printing. For example, at day 2, a printed rectangle lattice with a Young's modulus of 3.88 MPa could be easily bent and conformally adhered to a metal rod with a diameter of 1.26 mm, while similar prints became harder (9.83 MPa) at day 6 and remained planar on the top of the same metal rod (FIG. 3b). The rectangle lattice can also be repeatedly stretched without damage, demonstrating reliable junctions between filaments (FIG. 3c). Through a comparison to other 3D printed biopolymers (FIG. 3d and Table 2), the present disclosure demonstrates significantly improved mechanical properties for the 3D prints as well as an expanded range of tunability; of note is the absence of organic solvent and covalent crosslinking in the printing.

TABLE 2
Comparison of Mechanical properties between
typical and 3D printed materials.

| | Elastic Modulus | Ultimate | Ultimate |
|---|---|---|---|
| Materials | (kPa) | Stress (kPa) | Strain (%) |
| Alginate | 446 ± 72; | — | 42 ± 8 |
| | 15.5 ± 2 | | |
| | (compression) | | |
| 3D Printed | 50 | — | 21 |
| alginate | | | |
| Reinforced | 405 ± 68 | — | — |
| GelMA in | (compression) | | |
| 3D PCL scaffolds | | | |
| Wet-spinning | — | 450,000 | 27.7 |
| fibroin fiber | | | |
| Salt-leaching | 3330 ± 500 | 320 ± 10 | — |
| Sponge | (compression) | (compression) | |
| 3D HA-PCL | 4000-11000 | — | 32-67 |
| hyperelastic bone | | | |
| Current work | 1000-6000 | 500-800 | >35 |
| (unoptimized) | | | |

3D Protein-based prints can provide a structural basis for support, as well as for a variety of biofunctions (FIG. 3e). Bioactive molecules, antibodies, antibiotics and nanoparticles can be doped into the protein ink. The mild and aqueous process of 3D biomimetic printing preserves bioactivity, adding value for both the preservation and function of enzymes and other proteins within the silk matrix. For example, horseradish peroxidase (HRP) doped protein ink was printed (FIG. 3f) and the enzyme remained active for weeks. Another example is the doping of Quantum dots and fluorescent dyes (FIGS. 3g and 3h).

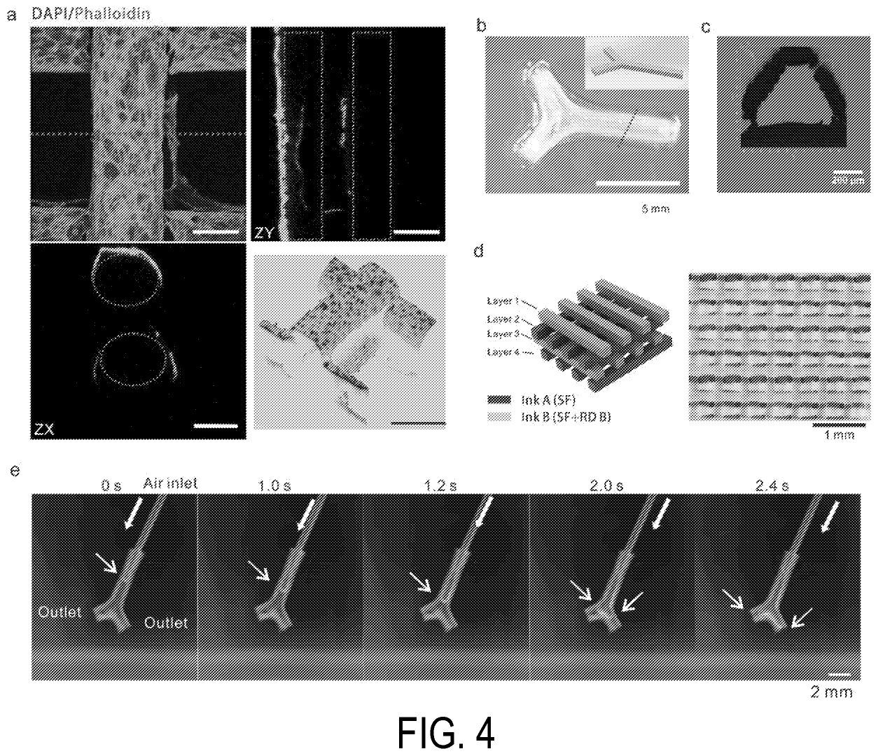

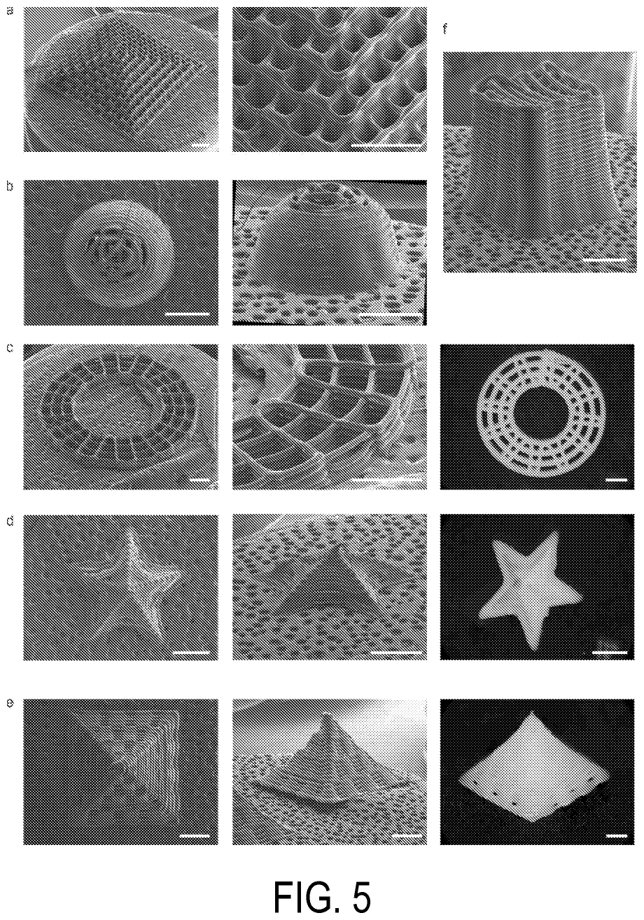

Degradability and biocompatibility may be useful features for protein structures, such as implants. The degradation profiles of different protein lattices are shown in FIG. 21. To demonstrate a more complex architecture, microfluidic chips were prepared using the 3D printing method (FIGS. 4b and c). The elasticity of the protein structures enabled the integration of active check valves, while also suggesting directions of utility for vascularized tissues with physiological functions like heart valves. Further, to investigate cell-compatibility, human umbilic vein endothelial cells (HUVECs, 2×10⁶ cells) were seeded on printed 4-layer lattices. After 5-days of culture, the HUVECs formed confluent monolayers of endothelialization (FIG. 4a). Immunostaining for ZO-1 showed the formation of tight junctions between cells. The printed structures that resembled decellularized tissues and cells were subsequently seeded after printing.

The silk article of the 3D printing method of the present disclosure can be selected from the group consisting of a degradable structure, a device, a system, a microfluidic chip, a hollow Y-shape tube, a blood vessel, a nerve conduit, an implantable scaffold, an optical lens, and combinations thereof.

### EXAMPLES

The following examples set forth, in detail, ways in which the flexible composite material may be synthesized, and will enable one of skill in the art to more readily understand the principles thereof. The following examples are presented by way of illustration and are not meant to be limiting in any way.

### Example 1

Preparation of Protein Ink and 3D Printing Procedure

The protein ink was prepared using 5 g of sliced cocoons from Bombyx mori (Tajima Shoji Co., Yokohama, Japan) were degummed in 2 L boiling solution of 0.02 M sodium carbonate for 30 min. The degummed fibers were dried overnight and solubilized in 9.3 M lithium bromide for 4 h in a 60° C. oven, followed by a dialysis (MWCO, 3,500) against DI water with 6 changes over 3 days. The insoluble particulates were removed by centrifugation (two times at 9,000 rpm, 20 min, 4° C.) and syringe filtration (low protein binding PVDF membrane, 5 μm, Merck Millipore, Ireland). The filtered solution was loaded into Slide-a-Lyzer dialysis cassettes (MWCO 3,500, Pierce) and concentrated by drying under 4° C. for 8 days to obtain high concentrations from 14 wt % to 30 wt %. Silk concentration was determined by weighing a dried sample of a known volume. The ink was doped with HRP, ampicillin or nanomaterials (Quantum dots and gold nanorods (10 nm, 808 mm, Sigma)) at certain ratios. When printing, the ink was loaded into a 3 ml syringe (EFD, Inc., RI, USA) quipped with a 33-gauge dispensing tip (EFD, Inc., RI, USA). 3D printing was performed with an Inkredible printer (Cellink, Sweden), coupled with an open-source software, Repetier host, and compressed air pump. All printing geometries were controlled by manually written G-code commands.

Morphology Characterization

Atomic force microscopy (AFM) was performed by using the Asylum Research Cypher AFM in an AC mode in air at scan rates of 2.44 or 0.8 Hz and aluminum coated silicon cantilevers (300 kHz and 40 N/m, nominal values) (Budget Sensors, USA). AFM images were processed by Gwyddion. Scanning Electronic microscopy (SEM) imaging was performed using an Ultra 55 field-emission SEM, Carl Zeiss AG, at an acceleration voltage of 5-8 kV. All SEM specimens were coated with a 5-10 nm thick Pt/Pd (80:20).

Rheology Characterization and Model Fitting

Rheology was performed on an ARES-LS2 (TA Instruments, New Castle, Del.) using a pair of 25 mm stainless steel parallel plates. To prevent evaporation during testing, mineral oil was placed around the plates. Dynamic strain sweep was performed to obtain the linear viscoelastic region (LVR). Dynamic time sweep chooses 1% strain from the LVR and is preformed to demonstrate 1) the stability of the ink; and 2) the solvent effect by pipetting the saline around the plates. The viscosity-shear rate curves were fitted using simplified Carreau-Yasuda model (Equation 1) and Hershel-Buckley model (Equation 2) at low and high shear rate regions, respectively:

$$\begin{matrix}η ( \dot{γ} ) = {{η}_{0} ( 1 + {( λ \dot{γ} )}^{a} )}^{\frac{n - 1}{a}} & ( 1 ) \\ η ( \dot{γ} ) = \frac{{τ}_{0}}{\dot{γ}} + {K ( \dot{γ} )}^{n - 1} & ( 2 )\end{matrix}$$

where viscosity (η) is a function of shear rate (\dot{γ}), η₀ is the zero-shear viscosity, TO is the yield stress, K is the consistency factor, α and n are indexes.

TABLE 3
Parameters of simplified Carreau-Yasuda (CY) and
Herschel-Bulkley (HB) models fitted on the viscosity-
shear rate profile of protein inks at different concentrations.

| | Silk | | | | Shear |
|---|---|---|---|---|---|
| | concentration | τ₀ (Pa) | K | n | rate (Pa S) |
| HB | 30% | 0.635 | 9.779 | 0.850 | 0.126-158.49 |
| model | 27% | 0.035 | 3.815 | 0.950 | |
| | 25% | 0.168 | 2.313 | 0.900 | |
| | 14% | 0.0924 | 0.204 | 0.939 | |

FTIR Characterization

The structures of the printing ink and prints were characterized by FTIR spectroscopy in ATR mode (Jasco FTIR-6200, Jasco Instruments, Easton, Md.). For each measurement, 64 times of scanning was utilized with a nominal resolution of 4 cm⁻¹. Spectral corrections and deconvolution were performed using a home-developed MATLAB package. The spectra were first smoothed with a 5-point triangle smoothing method and then baseline corrected using a cubic spline for the amide I band. The deconvolution was performed with a secondary derivative method. The secondary structure analysis was performed according to the literature (Guo et al. Biomacromolecules 2018, 19, 906-917)

Mechanical Testing

The mechanical tests of the dog-bone shaped protein prints were carried out by using an Instron 3366 machine (Instron, Norwood, USA) in tensile mode in a water cup with a tensile speed of 0.13 mm/s. The prints were griped at the expanded regions at both ends. The cross-section area was calculated in ImageJ based on SEM images of the prints that were manually snapped in liquid nitrogen.

In Vitro Enzymatic Degradation

The in vitro degradation of the 3D proteinaceous prints (in the shape of a ¾ sphere with 3 mm diameter) was evaluated using protease XIV (P8811, Sigma-Aldrich) with an activity of 6.7 U/mg. 3D prints were immersed in 5 ml of phosphate buffer saline (pH 7.4) containing protease (1U or 20 U) at 37° C. The enzyme solution was replaced with newly prepared solution every 24 h. After the specific time, samples were washed with phosphate buffer saline and distilled water, followed by lyophilization, weighing and SEM imaging. For controls, samples were immersed in phosphate buffer saline without enzyme.

Cell Culture and Imaging

Primary Human umbilical vein endothelial cells (HUVECs, C2519A, Lonza) were cultured in EGM™-2 BulletKit™ Medium (Lonza) to reach ˜80% confluence till passage 4. Then, HUVECs were harvested and seeded on the protein prints at 3×10⁶ cell/100 μl. After 5 days, the cells and prints were fixed and dyed with DAPI and Alexa Fluor™ 488 Phalloidin (Thermo Scientific, USA), followed by imaging with a Leica SP8 confocal microscope (Leica microsystems, Germany). Images were processed using ImageJ (NIH).

### Example 2

Preparation of Protein Ink and 3D Printing Procedure

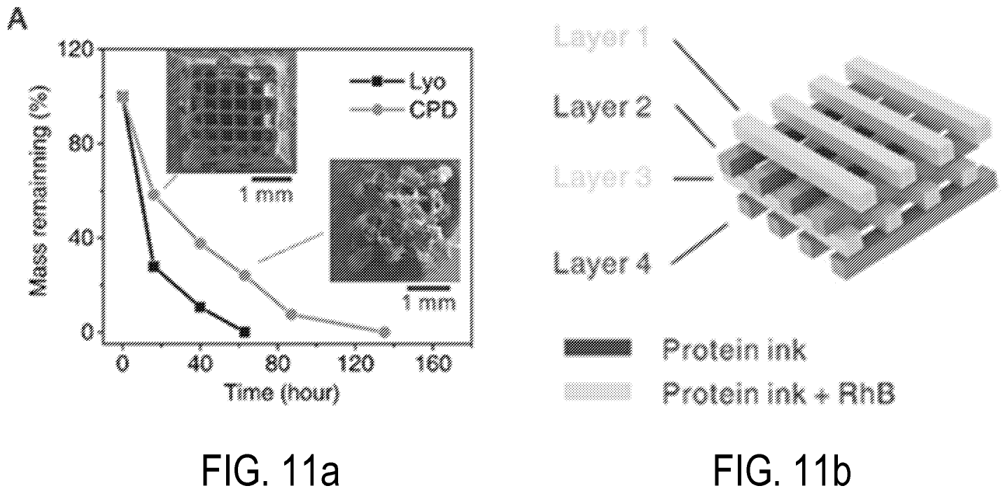

The protein ink was prepared using 5 g of sliced cocoons from Bombyx mori (Tajima Shoji Co., Yokohama, Japan) were degummed in 2 L boiling solution of 0.02 M sodium carbonate for 30 min. The degummed fibers were dried overnight and solubilized in 9.3 M lithium bromide for 4 h in a 60° C. oven, followed by a dialysis (MWCO, 3,500) against DI water with 6 changes over 3 days. The insoluble particulates were removed by centrifugation (two times at 9,000 rpm, 20 min, 4° C.) and syringe filtration (low protein binding PVDF membrane, 5 μm, Merck Millipore, Ireland). The filtered solution was loaded into Slide-a-Lyzer dialysis cassettes (MWCO 3,500, Pierce) and concentrated by drying under 4° C. for around 8 days to obtain high concentrations around 30 wt %. Silk concentration was determined by weighing a dried sample of a known volume. The ink was doped with horseradish peroxidase (HRP)-, labeled antibody (A0293, Sigma-Aldrich) in 1:1000, rhodamine B (79754, Sigma-Aldrich, saturated solution) in 1:100, Fluorescein (46955, Sigma-Aldrich, saturated solution) in 1:100 and Quantum dots (900225, Sigma-Aldrich) in 1:100 by stirring for 2 minutes and settling for 1-2 hours till all bubbles disappear. The solution of silk fibroin, as printing ink, was a viscous yellowish clear liquid with ˜6% β-sheet content, ˜30 wt % concentration, pH ˜7 and ˜90 kDa molecular weight, analogous to native silk protein dope except for the lower molecular weight (˜90 kDa vs. ˜300 kDa). The hydrodynamic diameter of the diluted ink was around 23 nm, suggesting a single molecular dispersion. Rheological characterization of the ink showed typical shear-thinning behavior (FIG. 11a). We further used the Herschel-Bulkley (HB) model to fit viscous-shear rate profiles at the region of high shear rate (>0.1 s⁻¹), as well as finite element analysis (FEA) to simulate the rheological behavior of the ink in the nozzle during 3D printing. At a printing pressure of 210 kPa, the viscosity decreased by nearly a hundred-fold (from 547.22 Pa·s to 6.18 Pa·s) and the simulated shear rate reached ˜65 s⁻¹, above in vivo critical values (˜1-10 s⁻¹), indicating the elongation of protein molecules during 3D printing, mimicking native silk spinning.

De Novo Aqueous Salt Bath

As the crux of this work, we rationally developed an aqueous bath with a de novo composition of inorganic salts (0.5 M dipotassium phosphate and 4 M sodium chloride) to recapitulate solvent conditions of silk spinning by two evolutionarily-distant species, spiders and silkworms. The chemical composition as well as the working mechanism to solidify ink of this bath is essentially different from others for wet spinning and 3D printing for proteins and polymers as shown in Table 4. First, the bath is composed of the most abundant salt ions in the spinning glands, such as potassium and sodium. We assume that the metal ions participate the spinning of silks perhaps by imposing specific effects on proteins through interacting with water molecules on protein surfaces other than forming ionic bonds. Second, the bath is of a slightly acidic pH (˜6) and high osmolarity (>8 M, as one sodium chloride molecule disassociates into two ions) to remove water from extruded ink, which recapitulates solvent conditions of acidification and dehydration for silk spinning. Notably, the concentration of ions at the site of silk fiber formation remains unknown, and dehydration via elevated osmolarity is indeed a general principle found in animals, for example in urine concentration. Third, the bath was optimized to tune the dynamics of molecular crosslinking\assembly, tightly related to the phase-transition of the ink from liquid to gel and characterized by changes of storage modulus (G′) (FIG. 11b). Two extremes of high (10 M) and low (zero) concentrations of potassium ions led to a dramatic increase (˜1,000-fold) of G′ that is prone to clog the nozzle and slight decrease (˜0.5-fold) that cannot support shape retention, respectively. So, these formulations and the consequent assembly dynamics are unfavorable for 3D printing. While the optimized bath with 0.5 M potassium resulted in a relatively moderate increase of G′ (˜14-fold from 1.05 Pa to 14.4 Pa within ˜2 minutes). Of note, this balanced dynamics led to shape retention, continuous extrusion and adherence of multiple layers, thus enabling the 3D printing of the protein ink. The balanced assembly dynamics also distinguishes this work from other state-of art fiber spinning.

TABLE 4
Comparison of fiber spinning and 3D printing techniques for alginate (polysaccharide)
and silk proteins.

| | | Alginate | Silk | Silk | Silk printing |
|---|---|---|---|---|---|
| | Silk spinning | printing | printing-A | printing-B | (This work) |
| Ink | Recombinant | 4% w/v | Glycidyl | 28%-30% | ~30% |
| composition | and | sodium | methacrylate- | Regenerated | Regenerated |
| | Regenerated | alginate | modified Silk | silk fibroin | silk fibroin |
| | silks | | fibroin | | |
| 3D printing | No | Yes | Yes | Yes | Yes |
| Technique | Fiber | Extrusion- | Digital light | Extrusion- | Extrusion- |
| | spinning | based 3D | processing | based 3D | based 3D |
| | | printing | | printing | printing |
| Mechanism | Protein | Ionically | Photo- | Protein | Biomimetic |
| | precipitation | crosslinking | crosslinking | precipitation | solvent effects |
| Crossing- | Isopropanol, | 11 mM | LAP | 95% | 4M NaCl + |
| linking | methanol or | CaCl₂ | photoinitiator | Methanol | 0.5 M K₂HPO₄ |
| Reagent | ~30% | | (under UV) | | |
| | ammonium | | | | |
| | sulfate | | | | |
| Tensile test | Yes | Yes | Yes | No | Yes |
| Mechanical | High | Low | Low | Not specified | High |

| | |
|---|---|
| performance | Details in Table 5 |

Ordered and Hierarchical Structure

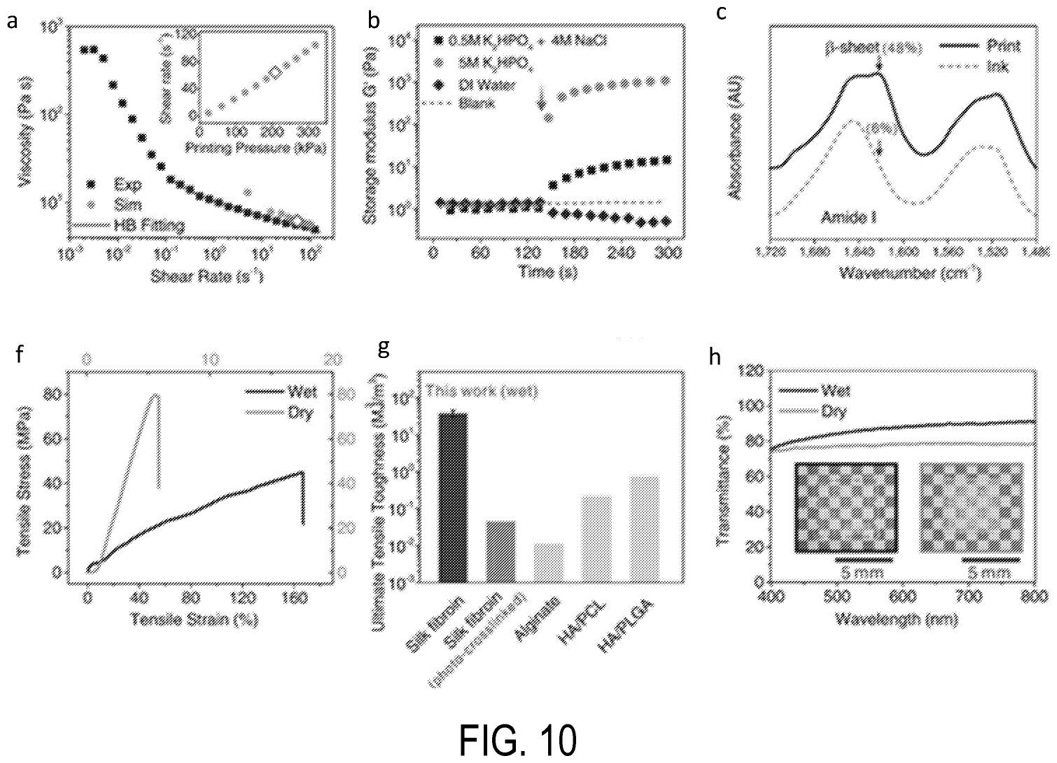

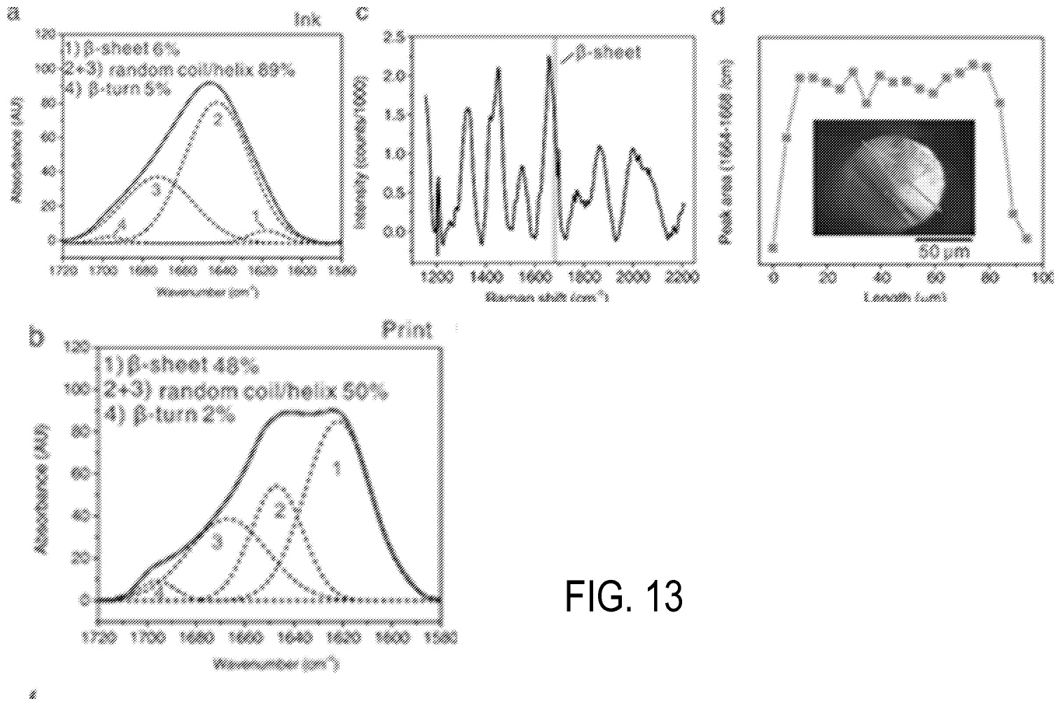

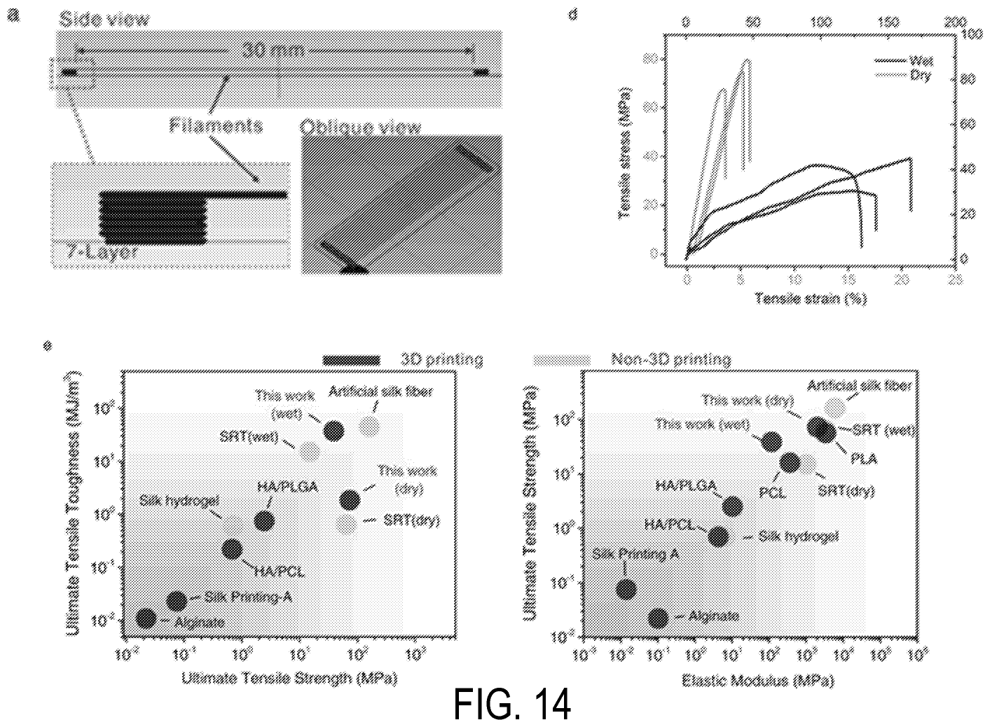

Ordered hierarchical organization of molecules is a characteristic of directed molecular assembly (FIG. 1). Fourier transform infrared spectroscopy (FTIR) spectra demonstrated the transformation from random coil/helix to β-sheet after printing with an approximate eight-fold increase from ˜6% to ˜48% (FIG. 10c), indicating that the silk fibroin molecules assembled through the stacking of the repetitive domain (GAGAGS) with the formation of hydrogen bonds, notable as physical crosslinks without chemical or photochemical crosslinking requirements. Scanning electron microscopy (SEM) images of the cross-sections of a filament, fractured by tensile force, showed that the assembled molecules aggregated into nanofibrils (˜200 nm in diameter) (FIG. 13d); the individual nanofibrils seem to be pulled out in these images. And SEM images of another filament, fractured in liquid nitrogen, showed nanofibrils densely packed into solids with an almost homogeneous morphology at the nanoscale. Sparse nanoholes in the cross-sections were observable and probably caused by residual water. The homogeneous molecular conformation was also confirmed by Raman spectral mapping across the full width of a filament, which showed the almost unvaried distribution of β-sheet and perhaps exclude a core-shell structure (FIGS. 13c and 13d). Moreover, birefringence images demonstrated longitudinal alignment of the protein molecular chains including both amorphous and crystalline regions (FIG. 13a-d).

Mechanic and Optic Performance

A 3D printed 4-layer lattice (in wet) is of compliance, extensibility and durable junctions between layers that remain intact under repeatedly stretching and folding. To characterize the mechanical performance in uniaxial tensile tests, single filaments of ˜30 mm in length were directly cut from a seven-layer 3D print without post-stretching (FIG. 11f, 14a). The prints demonstrate differential mechanical behaviors in dry and wet. Generally, water molecules make proteinaceous prints less stiff but more extensible. Ultimate tensile strength (39±8 MPa, wet) of the print outperformed that of both photo-crosslinked (0.075±0.0075 MPa, wet) and organic solvent-processed silk fibroin (0.08-0.7 MPa, wet) by 2-3 orders of magnitude (there is no tensile test results of 3D printed silk fibroin in methanol for comparison); moreover, ultimate tensile toughness (37±7 MJ/m³, wet) was higher than that of all other 3D printed polymers ranging from 0.2 to 27.6 MJ/m³ (FIG. 11g, 14f and Table 5). The toughness of the filament as part of 3D prints was superior to or comparable with natural and artificial fibers (native silkworm silk, ˜70 MJ/m³; flax, 7-14 MJ/m³; supramolecular fiber, 22.8±10.3 MJ/m³; and recombinant silk fiber, 45±7 MJ/m³). The high toughness rendered 3D prints capable of absorbing significant energy prior to facture, which is desired for prey trapping and athletic gears. Of note, the remarkable mechanical performance results from the ordered hierarchical structures, and highlights the distinctive capability of solvent-directed molecular assembly in comparison with, for example, methanol bath and chemical and photochemical crosslinking. In addition, the absence of intensive energy input (like temperature-induce phase transition) during 3D printing makes the resulting mechanical performance particularly compelling.

The dense morphology allows 3D prints visually transparent, such as the vase and lattice and membrane (FIG. 11h), promising for levitating bio-photonic applications to 3D. For measuring transmittance, we used 3D printed ˜0.1 mm-thick membranes composed of parallel and contiguous filaments (FIG. 11h). The transmittance in both dry and wet states was above 80% from 400 to 800 nm, comparable to previous results of proteinaceous structures fabricated by 2D manufacturing like spinning coating or film casting.

TABLE 5
Summary of stress-strain properties of biomaterials shaped by 3D printing and other
engineering methods (Mean value ± Standard error).

| | | | Tensile | Tensile | Tensile | Elastic |
|---|---|---|---|---|---|---|
| | | Sample | Toughness | Strength | Strain | Modulus |
| | Materials | Status | (MJ/m3) | (MPa) | (%) | (MPa) |
| Non-3D | Silk Fiber | Dry | 45 ± 7 | 162 ± 8 | 37 ± 5 | 6 ± 0.8 (GPa) |
| printing | Molded Silk Hydrogel | Wet | 0.003-0.6 | 0.08-0.7 | 3-127 | 1.9-6.5 |
| | Molded Sucker Ring | Wet | ~15 ^{a} | 25 | 60 | 1 (GPa) |
| | Teeth | Dry | 0.64 ^{a} | 64 | 2 | 3.3 (GPa) |
| | (True stress and strain) | | | | | |
| 3D | Alginate | Wet | 11 (kJ/m³) ^{a} | 22 (kPa) | ~22 | 102 ± 27 |
| printing | (kPa) | | | | | |
| | HA/PCL | Wet | 0.2 ^{a} | 0.7 ^{a} | 61.2 ± 6.4 | 4.3 ± 0.4 |
| | HA/PLGA | Wet | 0.8 ^{a} | 2.5 ^{a} | 36.1 ± 4.3 | 10.3 ± 1.3 |
| | Liquid Crystal polymer | Dry | 27.6 | 500 | 9% | 16 (GPa) |
| | (Vectra A950) | | | | | |
| | PCL | Dry | NS | 10.5-16.1 | 0.1-8 | 343.9-364.3 |
| | PLA | Dry | 1.6 | 56.6 | 1.93 | 3.4 (GPa) |
| | Silk printing-A | Wet | 23 (kJ/m³) ^{a} | 75 ± 7.5 (kPa) | 124 ± 41 | 14.5 ± 2.9 |
| | (Photo-crosslinking) | | | | | (kPa) |
| | Silk printing-B | Dry | NS | NS | NS | 5.6 ± 1.4 |
| | (Organic solvents) | | | | | (GPa) |
| | Silk printing | Wet | 37 ± 7 | 39 ± 8 | 128 ± 34 | 117 ± 71 |
| | (This work) | Dry | 2 ± 0.4 | 74 ± 6 | 5 ± 1 | 2 ± 0.3 (GPa) |

^{a} Estimated from published stress-strain curves;
PLGA, polylactic-co-glycolic acid;
PLA, Polylactic acid;
PCL, polycaprolactone;
HA, hydroxyapatite;
NS, not specified.

Biocompatible and Multi-Material Printing

The whole process of 3D printing is at ambient and aqueous conditions, which is highly desired to preserve the biodegradability and cytocompatibility as well as the integrated biofunctions of 3D proteinaceous prints, because harsh processing conditions and toxic organic solvent probably deteriorate these properties. The biodegradability was investigated in the proteolytic degradation of the prints (FIG. 11a). Two post-processing techniques including critical point drying (CPD) and lyophilization (Lyo) results in different degradation profiles, owing to different levels of protein crystallinity, thus offering an option to tune the degradation dynamics. The cytocompatibility was shown by the confluent layer of human umbilic vein endothelial cells, i.e. endothelization. The printing ink allows a wide range of additives, including quantum dots, small fluorescent molecules and especially bioactive horseradish peroxidase, to render 3D prints additional functions. The enzymatic activity of horseradish peroxidase integrated into a two-layer 3D print allows emitting in the presence of enzyme substrates. Of note, it is advantageous of this work to eliminate the use of methanol in comparison with 3D printing in methanol bath because methanol reduces the activity of horseradish peroxidase in a silk fibroin film by nearly 4-fold. Considering the well-established composite silk fibroin materials, we envision more additives such as graphene and antibiotics can be integrated into the proteinaceous 3D prints. Furthermore, the capability of programming multi-materials with special precision into functionally heterogeneous architectures is common in nature and nontrivial in many artificial applications, which was exemplified by printing an alternate pattern of fluorescence into 3D proteinaceous structures (FIG. 11b).

Microfluidic Channels

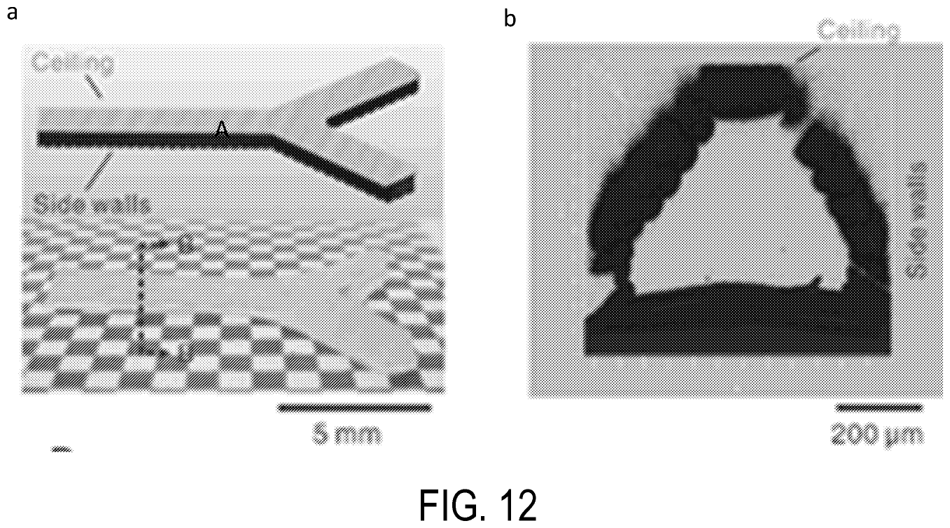

We further demonstrated the manufacturing capability by constructing a bifurcate and perfusable microfluidic channel in a single-step manner (FIG. 12). A structure like the microfluid channel places stringent demands on the printing capability; the printed structures are overhanging at the ceiling and high aspect-ratio at the sidewall. Besides, the two types of structures require rapid assembly dynamics to prevent subsidence and relatively slow assembling dynamics to form seamless and water-proof adherence between sequentially extruded filaments, respectively. Along with the optimized assembly dynamics, we addressed these demanding requirements by using compartmentalized priming parameters. For bottom layers and side walls, the printing speed and pressure was 1 mm/s and ˜210 kPa, respectively; while for building the ceiling layer, the printing speed and pressure was elevated to 1.5 mm/s and ˜250 kPa, respectively. Of note, this work eliminated the use of sacrificial and supporting materials to significantly streamline the manufacturing of complex and hollow shapes. The printed ten-layer microfluidic channels demonstrated high resolution (˜350 μm diameter and ˜100 μm wall thickness), elasticity, mechanical stability and desired perfusability. The channel thus can be reversibly bent over a large curvature and controlled with a pinch-based valve. The backpressure of the microfluidic channel was up to 300 kPa, which covers the range of most applications of microfluidics as well as physiological blood pressures, promising for constructing artificial vascular grafts. Indeed, the silk fibroin is particularly suitable for constructing small-diameter vascular grafts (<6 mm), because it unlike synthetic polymers such as poly-tetrafluoroethylene and poly(ethylene terephthalate) will not suffer from thrombus formation and intimal hyperplasia. Furthermore, 3D printing offers significant manufacturing flexibility in comparison with other techniques including soft lithography, spinning and coating for making microfluidic chips and proteinaceous vascular grafts.

Morphology and Polarized Optical Microscopy Characterization

3D prints were dried in a critical point dryer (CPD 300, Leica, Germany) and coated with a 5-10 nm thick Pt/Pd (80:20), followed by scanning electronic microscopy (SEM) imaging (Ultra 55 field-emission SEM, Carl Zeiss AG, Germany) at an acceleration voltage of 5 kV. The cross-section of 3D printed filaments was obtained from breaking in liquid nitrogen or tensile test. The cross-section of 3D printed microfluidic channels was imaged with a Leica SP8 confocal microscope using the autofluorescence of the assembled silk fibroin. The polarized optical microscopy (Eclipse E200POL, Nikon, Japan) equipped with a first order red retardation plate was used to image birefringence of 3D prints.

Rheology Characterization and Model Fitting

Rheology was performed on an ARES-LS2 (TA Instruments, New Castle, Del.) using a 25 mm stainless steel cone with an angle of 0.0994° and a gap of 0.0468 mm and a 50 mm stainless steel plain plate. Static strain sweeps were performed to obtain the viscosity-shear rate curve. Part of the curve (at shear rate >0.1 s⁻¹) was fitted with Herschel-Bulkley (HB) model, as shown below:

$$η ( \dot{γ} ) = \frac{{τ}_{0}}{\dot{γ}} + {K ( \dot{γ} )}^{n - 1}$$

where viscosity (η, Pa·s) is a function of shear rate (\dot{γ}, s⁻¹), τ₀ is the yield stress (Pa), K is a consistency factor (Pa·s¹⁻ⁿ), and n is an index. The fitted values of τ₀, K and n are 0.683 Pa, 9.707 Pa·s¹⁻ⁿ and 0.852, respectively.

Finite Element Analysis (FEA) was performed with COMSOL Multiphysics 5.3a (COMSOL Inc., MA) using the fitted parameters and experimentally measured flow rates at printing pressures ranging from 50 kPa to 300 kPa. A 2D symmetry model of the nozzle was established and meshed into 16,000 triangle elements. The results were reconstructed in 2D and 3D by mirroring and revolving, respectively. The quantitative data of viscosity and shear rate were calculated from averaging the value of each element in the whole simulated model.

We performed oscillatory time sweep at 1 Hz and 1% strain (within the linear viscoelastic region) for 5 min. At ˜140s, 100 μl of the printing bath, 5M dipotassium phosphate or DI water was pipetted around the circumstance of the cone for evaluating the dynamics of the molecular assembly.

Fourier Transform Infrared Spectroscopy (FTIR) Characterization

The secondary structures of proteins (β-sheet, random coil/helix and β-turn) of the printing ink and the prints (both lyophilized) were characterized by FTIR spectroscopy in ATR mode (FTIR-6200, Jasco Instruments, Easton, Md.) and analyzed by semi-qualitative spectral deconvolution of amide I band (1,580-1,720 cm⁻¹) according to previous reports. For each measurement, 64 scans were co-added with a nominal resolution of 4 cm⁻¹.

Raman Microscopy

The cross-sectional line profile of the printed filament was characterized with a confocal microscope equipped with a Horiba Multiline Raman Spectrometer (Horiba scientific, Japan). The Raman spectrometer contains 633 nm He—Ne diode laser (˜2 mW), 600 gr/mm grating and Synapse CCD detector. Raman spectrums were obtained at 20 points, separated by 5 μm, with exposure time of 30 seconds and twice accumulations at each point. Data acquisition was automatically denoised and controlled by Lab Spec 6 software (Horiba scientific, Japan). The peak area between 1664 cm⁻¹ to 1668 cm⁻¹ was used to estimate the content of β-sheet.

Morphology and Polarized Optical Microscopy

3D prints were dried in a critical point dryer (CPD 300, Leica, Germany) and coated with a 5-10 nm thick Pt/Pd (80:20), followed by scanning electronic microscopy (SEM) imaging (Ultra 55 field-emission SEM, Carl Zeiss AG, Germany) at an acceleration voltage of 5 kV. The cross-section of 3D printed filaments was obtained from breaking in liquid nitrogen or tensile test. The cross-section of 3D printed microfluidic channels was imaged with a Leica SP8 confocal microscope using the autofluorescence of the assembled silk fibroin. The polarized optical microscopy (Eclipse E200POL, Nikon, Japan) equipped with a first order red retardation plate was used to image birefringence of 3D prints.

Transparency

For light transmission measurements, visible spectra were taken using a vis/near-infrared fiber-optic spectrometer (USB-2000, Ocean Optics, USA). White light was propagated through the fiber to pass through a 3D printed membrane composed of parallel and conterminous filaments, and the transmitted light was coupled into a fiber tip guided to the spectrometer. The distance between the illumination source and the collection tip was fixed at 10 mm.

Mechanical Testing

Single filaments of ˜30 mm length obtained from air-dried seven-layer 3D prints were used for uniaxial tensile test with Instron 3366 (Instron, USA) at cross-head speeds of 0.13 mm/s (for wet samples) and 0.013 mm/s (dry). The use of the single filament isolates the influence of local orientation and filled density. All filaments were dry. For wet samples, we hydrated dried filaments in DI water for 5 minutes. The ends of the filament were clamped in a pair of pneumatic grips (2752-005, Instron). Tensile strength and toughness were calculated based on the cross-sectional area of the filaments was measured from SEM images after snapping in liquid nitrogen. Elastic modulus was calculated from the initial range of strain (5%-10% for dry samples and 0%-5% for wet ones).

The present disclosure has described one or more preferred embodiments, and it should be appreciated that many equivalents, alternatives, variations, and modifications, aside from those expressly stated, are possible and within the scope of the invention.

We claim:

1. A three-dimensional printing method for making a three-dimensional silk article, the method comprising:
a) selecting an article formation parameter set including one or more silk fibroin solution parameters, one or more solvent bath parameters, one or more shear force parameters, and one or more mapping parameters; and
b) iteratively introducing a silk fibroin solution into a solvent bath via a three-dimensional printing outlet, thereby forming the three-dimensional silk article,
wherein the silk fibroin solution, the solvent bath, and control of the iteratively introducing are based on the article formation parameter set, and
wherein step b) is free of photo-cross-linkers, chemical cross-linkers, and organic solvents.

2. The three-dimensional printing method according to any one of the preceding claims, wherein the method further comprises:
c) removing the three-dimensional silk article from the solvent bath; and
d) drying the three-dimensional silk article.

3. The three-dimensional printing method according to any one of the preceding claims, wherein step d) includes freeze drying or critical point drying.

4. The three-dimensional printing method according to any one of the preceding claims, wherein, following the drying of step d), the three-dimensional silk article has one or more of the following properties:
an elastic modulus between 0.1-10 GPa;
an ultimate stress between 1-500 MPa;
a tensile toughness of between 0.5-5.0 MJ/m³;
an ultimate strain of between 0.1-50%;
silk fibroin having a β-sheet content of greater than 5%;
silk fibroin having a β-turn content of less than 15%;
a transmittance of 80% or greater over the visual range; and
combinations thereof.

5. The three-dimensional printing method according to any one of the preceding claims, wherein prior to drying or in the absence of drying the three-dimensional silk article is wet and has one or more of the following properties:
an elastic modulus between 10-1000 MPa;
an ultimate stress between 0.1-100 MPa;
a tensile toughness of between 10.0-100.0 MJ/m³;
an ultimate strain of between 35-500%;
silk fibroin having a β-sheet content of less than 10%;
silk fibroin having a β-turn content of between 5-35%;
a transmittance of 80% or greater over the visual range; and
combinations thereof.

6. The three-dimensional printing method according to any one of the preceding claims, wherein the one or more silk fibroin solution parameters includes a parameter selected from the group consisting of silk fibroin concentration, silk fibroin molecular weight distribution, and combinations thereof.

7. The three-dimensional printing method according to any one of the preceding claims, wherein the silk fibroin concentration is between 10 wt % and 40 wt %.

8. The three-dimensional printing method according to any one of the preceding claims, wherein the one or more solvent bath parameters includes a parameter selected from the group consisting of a chemical composition of the solvent bath, a solvent bath pH, a soak time, and combinations thereof.

9. The three-dimensional printing method according to the immediately preceding claim, wherein the chemical composition of the solvent bath comprises one or more salts.

10. The three-dimensional printing method according to the immediately preceding claim, wherein the one or more salts are selected from the group consisting of sodium chloride, dipotassium phosphate, ammonium sulfate, and combinations thereof.

11. The three-dimensional printing method according to any one of claim 8 to the immediately preceding claim, wherein the chemical composition of the solvent bath includes a total salt concentration of between 500 mM and 10 M.

12. The three-dimensional printing method according to any one of claim 8 to the immediately preceding claim, wherein the chemical composition of the solvent bath includes a sodium chloride concentration of at most 5.0 M.

13. The three-dimensional printing method according to any one of claim 8 to the immediately preceding claim, wherein the chemical composition of the solvent bath includes a dipotassium phosphate concentration of at most 2.0 M.

14. The three-dimensional printing method according to any one of claim 8 to the immediately preceding claim, wherein the chemical composition of the solvent bath includes an ammonium sulfate concentration of at most 2.25 M.

15. The three-dimensional printing method according to any one of claim 8 to the immediately preceding claim, wherein the solvent bath pH is between 4 and 7.

16. The three-dimensional printing method according to any one of claim 8 to the immediately preceding claim, wherein the soak time is between 1 day and 5 days.

17. The three-dimensional printing method according to any one of the preceding claims, wherein the one or more shear force parameters include a parameter selected from the group consisting of one or more nozzle dimensions, injection velocity, and combinations thereof.

18. The three-dimensional printing method of claim 17, wherein the one or more nozzle dimensions includes an inner diameter of between 5 μm and 50 μm.

19. The three-dimensional printing method of claim 17 or 18, wherein the one or more mapping parameters include locations and volumes at which the silk solution is introduced in step b).

20. The three-dimensional printing method of any one of claim 17 to the immediately preceding claim, wherein the one or more mapping parameters include a nozzle movement speed.

21. The three-dimensional printing method of the immediately preceding claim, wherein the nozzle movement speed is between 0.1 and 10.0 mm/s.

22. The three-dimensional printing method of any one of claim 17 to the immediately preceding claim, wherein the one or more mapping parameters are selected in step a) by converting a three-dimensional digital image file into the one or more mapping parameters.

23. The three-dimensional printing method according to any one of the preceding claims, wherein the solvent bath is at room temperature.

24. The three-dimensional printing method according to any one of the preceding claims, wherein the silk fibroin solution further comprises an additive.

25. The three-dimensional printing method of claim 24, wherein the additive is selected from the group consisting of a mammalian cell, a bioactive molecule, an antibody, an antibiotic, a nanoparticle, a dye, and combinations thereof.

26. The three-dimensional printing method of the immediately preceding claim, wherein the additive is the mammalian cell.

27. The three-dimensional printing method of claim 25 or 26, wherein the mammalian cell comprises a human umbilic vein endothelial cell (HUVEC).

28. The three-dimensional printing method of any one of claim 25 to the immediately preceding claim, wherein the additive is the bioactive molecule.

29. The three-dimensional printing method of the immediately preceding claim, wherein the bioactive molecule comprises horseradish peroxidase.

30. The three-dimensional printing method of any one of claim 25 to the immediately preceding claim, wherein the additive is the antibiotic.

31. The three-dimensional printing method of the immediately preceding claim, wherein the antibiotic comprises ampicillin.

32. The three-dimensional printing method of any one of claim 25 to the immediately preceding claim, wherein the additive is the nanoparticle.

33. The three-dimensional printing method of the immediately preceding claim, wherein the nanoparticle is selected from the group consisting of a gold nanoparticle, a quantum dot, and combinations thereof.

34. The three-dimensional printing method of any one of claim 25 to the immediately preceding claim, wherein the additive is the dye.

35. The three-dimensional printing method of the immediately preceding claim, wherein the dye comprises a fluorescent dye.

36. The three-dimensional printing method according to any one of the preceding claims, wherein the three-dimensional silk article is selected from the group consisting of a degradable structure, a device, a system, a microfluidic chip, a hollow Y-shape tube, a blood vessel, a nerve conduit, an implantable scaffold, an optical lens, and combinations thereof.

37. A three-dimensional silk article formed by the method of any one of the preceding claims.

38. A three-dimensional bio-printer configured to perform the method of any one of the preceding claims.
